(19) United States
(12) Patent Application Publication
(10) Pub. No.: US 2007/0166729 A1
(43) Pub. Date: Jul. 19, 2007

(54) Method and reagent for sequencing

(75) Inventors: Hideki Kambara, Hachioji (JP); Guohua Zhou, Nanjing (CN); Tomoharu Kajiyama, Higashiyamato (JP); Shigeya Suzuki, Tokyo (JP)

Correspondence Address: Stanley P. Fisher;Reed Smith LLP, Suite 1400, 3110 Fairview Park Drive, Falls Church, VA, 22042-4503, US

(73) Assignee: Hitachi, Ltd.

(21) Appl. No.: 11/542,246

(22) Filed: Oct. 4, 2006

(30) Foreign Application Priority Data
Oct. 4, 2005 (JP) 2005-291185

(51) Int. Cl. C12Q 1/68; C12Q 1/66

(52) U.S. Cl. 435/6; 435/8

(57) ABSTRACT

The present invention provides: a method for nucleic acid analysis including the steps of subjecting a reaction solution containing a sample nucleic acid to complementary strand synthesis with the sample nucleic acid as a template, reacting pyrophosphate produced in the complementary strand synthesis with 30 to 800 μM AMP in the coexistence of pyruvate phosphate dikinase to produce ATP, performing luciferase reaction with the ATP as a reaction substrate, and detecting chemiluminescence generated in the luciferase reaction to determine the presence or absence of the complementary strand synthesis; and a kit therefor.

### CLAIM OF PRIORITY

The present application claims priority from Japanese Application JP 2005-291185 filed on Oct. 4, 2005, the content of which is hereby incorporated by reference into this application.

### BACKGROUND OF THE INVENTION

1. Field of the Invention

The present invention relates to a method for nucleic acid analysis and a kit for nucleic acid analysis. More particularly, the present invention relates to DNA sequencing utilizing DNA complementary strand synthesis and to a kit therefor.

2. Background Art

DNA sequencing widely used employs gel electrophoresis and fluorescence detection. In this method, an analyte DNA fragment to be sequenced is first amplified. Subsequently, DNA fragments of various lengths from the 5′ end of the amplified DNA fragments are prepared, and their 3′ ends are fluorescently labeled to give different wavelengths according to base species. Each fluorescently labeled fragment was differentiated from the others by gel electrophoresis depending on their lengths differing by one base, while the base species at the 3′ ends are determined from fluorescent colors emitted by each fragment group. Since DNA fragments pass through the fluorescent detection portion in ascending order of length, the terminal base species of the DNA fragments are determined in the order of length from shortest to longest by measuring the fluorescent colors, and sequencing can thus be accomplished. Fluorescent DNA sequencers exploiting this method have been diffused widely and were used actively in the human genome analysis.

In 2003, the completion of the human genomic sequence analysis was declared, heading into the age of utilization of sequence information in medical or other various industries. The determination of the sequence of a particular region of interest often suffices for recent DNA analysis without the need for analyzing the whole long sequence. Therefore, convenient methods or apparatuses suitable for such analysis of short DNA sequences have been required.

One of techniques developed in response to these demands is sequencing by step-by-step complementary strand synthesis reaction typified by pyrosequencing. In this method, complementary strand synthesis reaction is performed by hybridizing primers to a targeted DNA strand and sequentially adding one by one four nucleic acid substrates for complementary strand-synthesis (dATP, dCTP, dGTP, and dTTP) to the reaction solution. Complementary strand synthesis reaction, if any, generates pyrophosphate (PPi) as a by-product thereof. In pyrosequencing, PPi reacts with APS (adenosine 5′-phosphosulfate) in the coexistence of ATP sulfurylase to produce ATP. This ATP reacts with luciferin in the coexistence of luciferase to generate luminescence. Thus, the complementary strand-synthesizing nucleic acid substrates added are confirmed to be incorporated into the DNA strand by detecting the generated luminescence, and the sequence of the targeted DNA strand can thus be determined (Electrophoresis 2001, 22, p3497-3504). The complementary strand-synthesizing nucleic acid substrates that have not been used in the reaction are immediately degraded with enzymes such as apyrase to avoid the influence on subsequent reaction steps. Of the complementary strand-synthesizing nucleic acid substrates, dATP is, however, structurally similar to a luminescent substrate ATP and causes background light due to its reaction with luciferin. Therefore, dATP derivatives such as dATPαS, which do not serve as luminescent substrates, are used instead of ATP.

On the other hand, methods with better cost performance have been demanded as DNA analysis techniques have been used widely. The use of a convenient and inexpensive apparatus and the reduction of reagent cost are important for reducing cost required for DNA sequencing. An inexpensive chemiluminescence detection apparatus can be realized by using a photodiode detector (Measurement Science and Technology 13 (2002) p1779-1785). However, the conventional method requires large amounts of DNA samples and reagents for DNA sequencing using this apparatus. Namely, since the chemiluminescence detection apparatus using photodiode has the detection limit with detection sensitivity lower by an order of magnitude than that of a system using photomultipliers, the amounts of luminescent reagents such as luciferase must be increased for the detection of fmol order ATP.

Pyrosequencing detects chemiluminescence derived from ATP produced from the reaction of PPi produced in DNA complementary strand synthesis with APS in the presence of ATP sulfurylase. However, if there exists background light, the obtained detection sensitivity does not always reflect the amount of ATP generated. For example, APS, though having approximately 1/1000 of reaction efficiency of ATP, reacts as a substrate with luciferase to emit light. To efficiently convert PPi produced in complementary strand synthesis to ATP, approximately 5 μM APS must usually be added to the reaction solution. When the volume of the reaction solution is set to 100 μl, 5 μM APS generates luminescence corresponding to approximately 500 fmol ATP. Thus, DNA sequencing with high precision by pyrosequencing must employ a DNA sample of a picomole level and large amounts of reagents commensurate therewith.

The amount of luminescence is increased with increases in luciferase concentration. Therefore, this means is effective for improving sensitivity in the detection of a trace amount of ATP and however, is not quite effective for pyrosequencing because background light caused by impurities in APS or other reagents also gets stronger. Alternatively, when the amount of APS used is decreased, a signal to be measured is also decreased. Therefore, as long as APS is used, the detection limit can not be reduced and is completely determined by APS concentrations.

In relation to these problems, a method using pyruvate phosphate dikinase (PPDK) is known as a technique for producing ATP from PPi without the use of APS. For example, luminometric assay by the conversion of PPi produced during PCR to ATP using AMP and PPDK and subsequent luciferin-luciferase reaction (K. Karasawa, et al., The Proceedings of Annual Meeting of The Pharmaceutical Society of Japan (2003), Presentation No. 29[P1]-1-204) and SNP analysis by the measurement of PPi based, on bioluminescence using PPDK and AMP (E. Munakata, et al., The Proceedings of Amuual Meeting of The Pharmaceutical Society of Japan (2004), Presentation No. 29[P2]-1-311) have been reported. A bioluminescent reagent and quantitative determination methods for adenosine phosphate ester and for a substance involved in an ATP conversion reaction system using the reagent have been disclosed (JP Patent Publication (Kokai) No. 09-234099A). However, all of these approaches are practiced using DNA samples in large amounts. A method enabling the analysis or sequencing of a trace amount of a DNA sample by use of PPDK and AMP is still unknown.

An object of the present invention is to provide a method for highly sensitive, convenient DNA sequence analysis with a trace amount of a DNA sample by removing a cause of background light presenting a problem for pyrosequencing.

### SUMMARY OF THE INVENTION

To attain the object, in the present invention, AMP that does not serve as a substrate for luciferin reaction is reacted with pyrophosphate (PPi) in the presence of PPDK to produce ATP. Substances contained in the reagents, which cause background light are identified, and background lights from these identified substances are reduced by using enzymes such as PPase. However, since four enzymatic reactions in sequencing using chemiluminescence (pyrosequencing) competitively occur in one reaction vessel, conditions need to be optimized in consideration of these reactions. For example, a reaction solution pH and an AMP concentration that influences some reactions (AMP is structurally similar to dATP etc., and therefore inhibits, if coexisting in large amounts, DNA complementary strand synthesis reaction) must be optimized. Thus, the present inventors have successfully achieved a method for highly sensitive sequence analysis by determining optimum conditions for applying ATP production reaction with AMP and PPDK to pyrosequencing.

For ATP, it is only necessary to remove it once after production reaction, while PPi is generated again by the degradation of dNTP. The present inventors confirmed that highly sensitive measurement can be achieved by adding in advance a trace amount of PPase (to a degree that does not affect sequencing) to reagents to degrade PPi prior to measurement. Background light could be reduced drastically by using AMP that does not serve as a luminescence substrate for ATP production reaction from PPi. This enabled sequencing using DNA at or below 0.1 pmol, which is an order of magnitude smaller than the DNA amount conventionally used. Moreover, sequencing performed by repetitively adding nucleic acid substrates to continuously perform complementary strand synthesis was accomplished by limiting the concentration of coexistent AMP to a particular region and thereby preventing other reaction inhibitions. This enabled drastic reduction in the amount and cost of reagents used in DNA sequence analysis.

As described above, in the present invention, highly sensitive DNA detection is realized, wherein ATP production reaction using AMP that does not serve as a substrate for luciferin reaction as well as the degradation of ATP or PPi contained in reagents is performed, thereby removing background light attributed thereto. The improvement of detection sensitivity by the present invention allows for DNA testing equipment using inexpensive photodetectors, micro DNA analysis devices for the DNA sequencing of a trace amount of DNA accommodated in fine reaction cells, and DNA sequencing using large-scale DNA analyzers using many reaction cells, and achieves efficient DNA analysis.

### BRIEF DESCRIPTION OF THE DRAWINGS

FIG. 1 shows the principle of DNA sequencing based on step-by-step complementary strand synthesis reaction (upper panel) and the outline of a method of the present invention using AMP and PPDK in ATP production reaction from PPi (lower panel);

FIG. 2 shows the outline of conventional pyrosequencing using APS in ATP production reaction from PPi;

FIG. 3 shows a specific example of sequencing using the method of the present invention. In the diagram, one-base, two-base, and three-base levels indicate the number of a base incorporated at a time per DNA;

FIG. 4 shows the pH dependence of luciferase and PPDK activities;

FIG. 5 shows the temperature dependence of luciferase and PPDK activities;

FIG. 6 is a bird's eye view of a small-size apparatus for DNA sequencing;

FIG. 7 is a graph showing the relationship between AMP concentrations and signal intensity variations obtained in luciferase luminescence reaction with ATP produced using PPi obtained in DNA complementary strand synthesis. The horizontal axis of the graph indicates AMP concentrations in the reaction solution;

FIG. 8 shows the comparison of signal intensity obtained by the method of the present invention with signal intensity obtained by the conventional method. As can be seen from the diagram, the method of the present invention produced overwhelming smaller background light;

FIG. 9 is a graph showing signal intensity and background light signal intensity variations with changes in luciferase amount;

FIG. 10 is a graph showing a result of sequencing a trace amount of a DNA sample (PCR amplification product of TPMT gene) by the method of the present invention;

FIG. 11 is a graph showing signal intensity variations with changes in temperature condition; and

FIG. 12 gives a summary of the result of FIG. 11.

### DETAILED DESCRIPTION OF THE PREFERRED EMBODIMENTS

The present invention relates to a method for nucleic acid analysis comprising the steps of subjecting a reaction solution containing a sample nucleic acid to complementary strand synthesis with the sample nucleic acid as a template, reacting pyrophosphate (PPi) produced in the complementary strand synthesis with 30 to 800 μM AMP in the coexistence of pyruvate phosphate dikinase (PPDK) to produce ATP, performing luciferase reaction with the ATP as a reaction substrate, and detecting chemiluminescence generated in the luciferase reaction to determine the presence or absence of the complementary strand synthesis.

It is more preferable that PPi should react at the ATP production step under conditions of an AMP concentration of 50 to 600 μM.

The complementary strand synthesis step may be performed by sequentially adding one by one four nucleic acid substrates or derivatives thereof corresponding to bases A, G, T, and C or simultaneously adding two or more of them. The sample nucleic acid can be sequenced on the basis of the presence or absence of the complementary strand synthesis.

dNTP as well as ddNTP can be used as the nucleic acid substrate. Examples of the derivatives include dATPαS and ddATPαS.

After complementary strand synthesis reaction, the redundant nucleic acid substrates or derivatives thereof hinder the measurement. It is therefore preferred that they should be removed immediately by enzymatic degradation. Examples of enzymes used can include apyrase and pyrophosphatase (PPase).

It is preferred that the pyruvate phosphate dikinase and/or the luciferase should be thermostable enzymes that stably function at 40° C. or higher (e.g., commercially available luciferases including Luciferase manufactured by Sigma and LUC-H 61314 with extractant resistance, LUC-C 61313 with high specific activity, and LUC-T 61315 with thermostability manufactured by Kikkoman, preferably LUC-T 61315 with thermostability manufactured by Kikkoman). For example, a Klenow fragment lacking exonuclease activity is preferable as DNA polymerase used in the complementary strand synthesis.

It is preferred that the reaction solution should be adjusted to within a pH range of 7.0 to 8.0 and a temperature range of 30 to 45° C. in light of enzyme activity and complementary strand synthesis reaction.

It is further preferred that PPi or ATP in the reagents causing background light should be degraded and removed by adding in advance a trace amount of an enzyme such as pyrophosphatase to the reagents.

The present invention also provides a kit for the method for nucleic acid analysis.

This kit comprises

1) DNA polymerase,
2) four nucleic acid substrates or derivatives thereof corresponding to bases A, G, T, and C,
3) AMP, phosphoenolpyruvate, and pyruvate phosphate dikinase, and
4) luciferase and luciferin,
and comprises an indication stating that “AMP is reacted at a concentration of 30 to 800 μM in a reaction solution with pyruvate phosphate dikinase”. In this context, the indication may be attached as an instruction to the kit. Alternatively, it may be described on the package surface or affixed in a sticky label form to the package surface. Moreover, the indication is not limited to the expression described here or terms used here, and different expression or terms may be used without departing from the contents or effects thereof.

It is desirable that the reagents such as enzymes contained in the kit for nucleic acid analysis of the present invention should be provided and used within the preferable ranges described above.

### EXAMPLES

Sequencing using chemiluminescence is performed by step-by-step reaction where one stage of sequencing of one base is completed in 30 to 60 seconds. Namely, four nucleic acid substrates are sequentially added to a reaction solution to detect the presence or absence of chemiluminescence observed. Injected reagents must be removed or degraded before next reagent injection. This is because residual injected reagents present problems such as inaccurate sequencing results and the proceedings of complementary strand synthesis differing from one target DNA strand to another. On the other hand, uniform reaction in a short time requires the agitation of the solution. Therefore, agitators such as vibration motors are attached to reaction cells. If the amount of background light is large, it is required that the background light should be measured in advance before reagent dispensation and subtracted from the amount of luminescence attributed to reaction. However, background light is largely swayed by the driven agitation motor. Therefore, the accurate determination of background light is sometimes difficult during the use of the agitation motor. Thus, it is desirable for measurement with high precision that a signal derived from background light should have 1/10 of signal intensity attributed to DNA to be measured.

When sequencing is performed with 0.1 pmol DNA sample, which is 1/10 of the DNA sample amount (1 pmol) conventionally used, the amount of background light must be equal to or lower than the amount of chemiluminescence emitted by 0.01 pmol ATP. The conventional pyrosequencing using the process of ATP production by the reaction of APS (adenosine 5′-phosphosulfate) with PPi uses APS as a substrate for luciferase reaction and therefore could not sequence a trace amount of DNA. APS and PPi are converted to ATP by an enzyme ATP sulfurylase. In this process, an excess of APS and the enzyme seem to first form complexes and subsequently react with PPi. The Michaelis constant of reaction for forming the complexes of APS and the enzyme has been reported to be Km=0.56 μM. Accordingly, approximately 5 μM or more APS is required for producing the complexes in sufficient amounts. When the volume of the reaction solution is set to 100 μl, the necessary amount of APS is 500 pmol. APS, when used as a substrate for luminescent reaction catalyzed by luciferase, has been reported to have 0.6×10⁻³ activity to ATP. Under the conditions of pyrosequencing, the efficiency of the luminescence process with ATP as a substrate is reduced due to the coexistence of apyrase, an enzyme that degrades dNTP and ATP. In consideration of these circumstances, chemiluminescence intensity attributed to APS is very large and is on the order of 0.1 pmol in terms of ATP. This intensity remains large even if the reaction volume is set to as small as 30 μl.

For reducing such background light, it is necessary to use the process of ATP production from PPi using a reagent that does not serve as a substrate for chemiluminescence catalyzed by luciferase is required. This achieved sequencing using a DNA sample amount smaller by one to two orders of magnitude than that conventionally used or sequencing using an inexpensive apparatus.

In Example below, a DNA sequencing method performed by converting PPi produced in DNA complementary strand synthesis to ATP by the action of an enzyme PPDK and detecting chemiluminescence by luciferase will be described as an example of such a process. However, the present invention is not intended to be limited to this Example.

### Example 1

1. Outline and Principle

In this Example, a DNA sequencing method performed by converting pyrophosphate (PPi) produced in DNA complementary strand synthesis to ATP by the action of PPDK and detecting chemiluminescence generated from chemiluminescence reaction using luciferase was investigated.

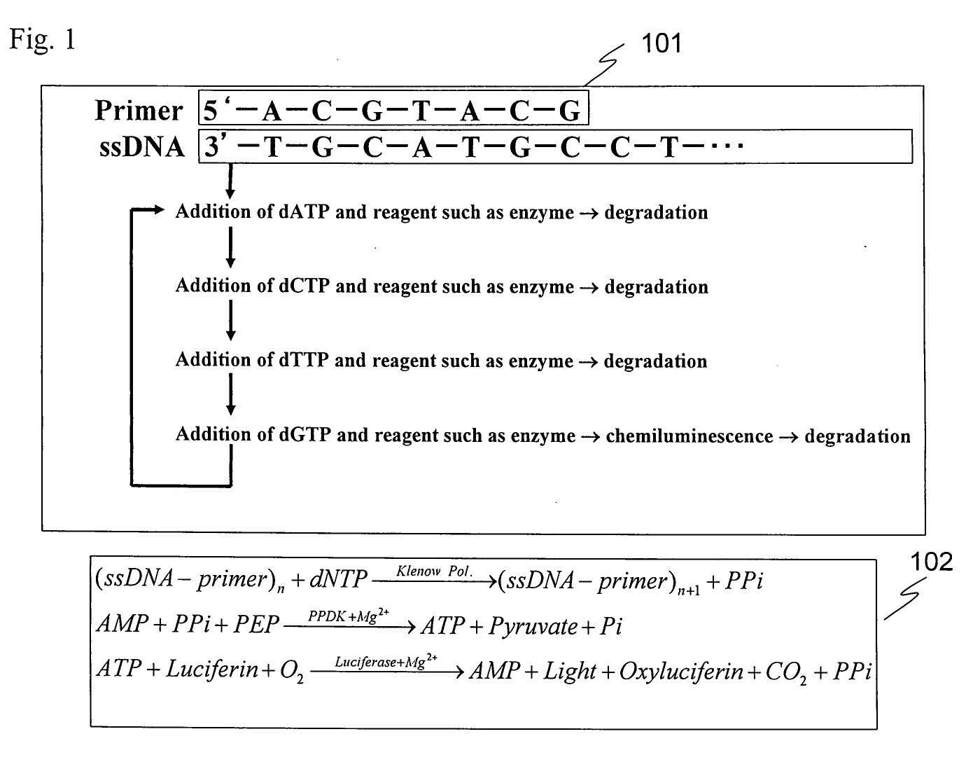

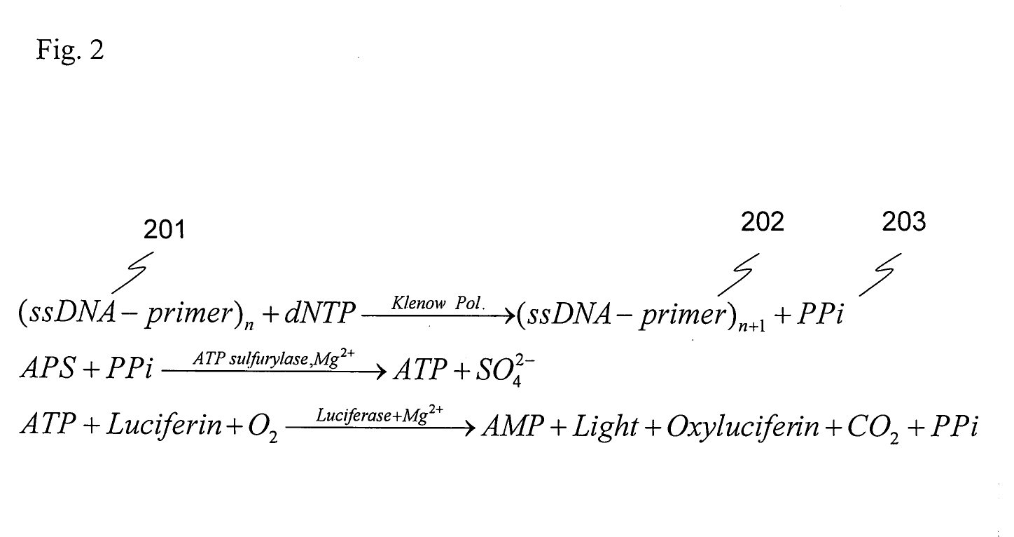

Enzymes used were DNA polymerase (EXO-Klenow, Ambion, Austin, Tex., USA, Cat #2008), PPDK (Kikkoman, PPDK-E 61317), apyrase (Apyrase from Potato, Sigma, St Louis, Mo., USA, A6410), and luciferase (Luciferase, Sigma, St Louis, Mo., USA, L1759). This reaction system is complicated, wherein four enzymatic reactions simultaneously proceed, and the substances involved in the reactions are correlated. The outline of the reaction of the present invention is shown in FIG. 1. Moreover, the outline of conventional pyrosequencing reaction using APS (adenosine 5′-phosphosulfate) is shown in FIG. 2.

In the conventional pyrosequencing, ATP production reaction from PPi has been carried out by enzymatic reaction using APS and ATP sulfurylase, as shown in the following equation and FIG. 2:

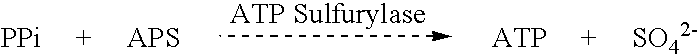

However, APS, though having approximately 1/1000 of reaction efficiency of ATP, reacts as a luminescent substrate for luciferase reaction with luciferin to generate chemiluminescence. Therefore, sequencing using a trace amount of DNA did not work in some cases due to background light attributed to APS.

In the method of the present invention, ATP production reaction from PPi is performed by reacting AMP that does not serve as a luminescent substrate for luciferase reaction with PEP in the presence of PPDK, as shown in the following equation and FIG. 1:

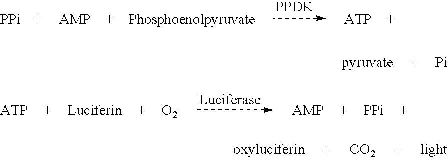

Since PEP used in the reaction and the resulting reaction product pyruvate do not serve as substrates for luciferase reaction, highly sensitive measurement with reduced background light can be achieved. As a result, the method of the present invention can provide sequencing using a DNA sample amount smaller by an order of magnitude than that in the conventional method.

The large difference between the method of the present invention and the conventional method is in that in the APS-based conventional system, APS utilized in ATP production is merely consumed and never reproduced and generates chemiluminescence by its reaction with luciferin in the presence of luciferase, whereas in the PPDK-based system of the present invention, the reaction substrate AMP does not react with luciferin. Therefore, the method of the present invention enables highly sensitive DNA sequencing by suppressing background light.

However, reagents including AMP used in the reaction often contain impurities such as ATP and PPi, which also give background light and cause reduction in detection sensitivity. Therefore, it is desired that these reagents should be supplemented prior to use with apyrase for ATP degradation or with PPase for PPi degradation and then used. However, these enzymes influence each reaction of pyrosequencing using the reagents. A possible approach for removing such influence in use is to remove the added enzymes after enzymatic treatment. However, since AMP, when remains left, is gradually degraded to accumulate compounds that affect the luminescence cycle, the enzymes must be removed immediately before the use of the reagents, leading to complicated experimental procedures. Thus, it was decided that before the use of or during the storage of reagents including AMP, a trace amount of degrading enzymes are added to the reagents to a degree that does not influence pyrosequencing, to perform enzymatic degradation over a long time (1 hour or more). In this Example, PPi contained beforehand in reagents was degraded by approximately one-day treatment by adding thereto PPase adjusted to 1 U/L. If the amount of the enzyme added is too large, its influence on measurement is increased. Therefore, the preferable amount of the enzyme added is approximately 10 U/L or lower.

The Michaelis constants (Km) of PPDK and AMP used have been reported to be 5 μM. Efficient reaction requires the coexistence of 50 μM or higher AMP. The process of conversion of PPi to ATP bottlenecks reactions in pyrosequencing. From this point in view, the use of a high concentration of AMP is desirable.

2. Material (Enzyme)

(1) Thermostable PPDK

The properties of thermostable PPDK used in this Example are shown in Table 1. Moreover, the standard protocol of its use and the composition of reaction reagents are shown in Table 2. This protocol is specified as a reference for using PPDK and is required to optimize the composition of reagents described below in detail according to contents when several enzymes are used together.

TABLE 1
Properties of thermostable PPDK

Properties

| | |
|---|---|
| Molecular weight | ca. 230 kDa (gel filtration) |
| Structure | 2 subunits of ca. 91 kDa (SDS-PAGE) |
| Michaelis constants | 5.0106 M (AMP) |
| | 3.8105 M (PPi) |
| | 2.8104 M (phosphoenolpyruvate) |
| | 2.0104 M (ATP) |
| | 1.3104 M (pyruvate) |
| pH optimum | 6.5-7.0 |
| pH stability | 6.0-11.0 |
| Optimum temperature | 55-60° C. |
| Thermal stability | at or below 55° C. |

Stable also at low temperature and tolerant to freeze-thawing

| | |
|---|---|
| Activators | Mg²⁺, Mn²⁺, CO₂ |
| Inhibitors | Zn²⁺, Hg²⁺, Ag⁺ |
| Specificity | AMP (100), UMP (0), IMP (0), TMP (0), |
| | CMP (0), GMP (0) |

TABLE 2
Thermostable PPDK use protocol

<Reagents>

| | |
|---|---|
| A. | Substrate solution: |
| | Dissolve 330 mg of (NH₄)₂SO₄, 89.2 mg of Na₄P₂O₇•10H₂O, |
| | 46.8 mg of phosphoenolpyruvate.Na₃•H₂O, 25 μl |
| | of 2-mercaptoethanol, 2.0 ml of 5.0 mM AMP•Na₂, and 10 ml |
| | of 0.5 M Bis-Tris propane-HCl buffer (pH 6.8) in 80 ml of |
| | distilled water, adjust to pH 6.8 with 2N HCl, and dilute with |
| | distilled water to 100 ml. (Store at 20° C.) |
| | Add 30 μl of 1.0 M MgSO₄ solution to 10 ml of the substrate |
| | solution before use. |
| B. | ATP standard solution, 2107 μmol/ml: |
| | Dilute CheckLite ATP standard (2106 M) (manufactured by |
| | Kikkoman) to 10000-fold volume with distilled water. |
| C. | Enzyme dilution buffer: |
| | Dissolve 0.5 g of bovine serum albumin (BSA), 62.5 μl of |
| | 2-mercaptoethanol, and 50 ml of 0.5 M Bis-Tris propane buffer |
| | (pH 6.8) in 400 ml of distilled water, adjust to pH 6.8 with |
| | 2N HCl, and dilute with distilled water to 500 ml. |
| | (Store at 20° C.) |

<Sample>

Dilute the enzyme preparation to a volume activity of
10⁴-10² U/ml in ice-cold enzyme dilution buffer (Reagent C)
immediately before measurement.

<Procedure>

| | |
|---|---|
| 1. | Add 0.18 ml of substrate solution (Reagent A) to a test tube. |
| 2. | Equilibrate at 37° C. for approximately 5 minutes. |
| 3. | Add 0.02 ml of sample and incubate at 37° C. for 30 minutes. |
| 4. | Allow to stand for 3 minutes in boiling water to stop the |
| | reaction. |
| 5. | Centrifuge the reaction mixture. |
| 6. | Dilute the supernatant of the reaction mixture to 10000-fold |
| | volume with distilled water. |
| 7. | Add 0.1 ml of the diluted reaction mixture to 0.1 ml of |
| | CheckLite 250 (manufactured by Kikkoman). |
| 8. | Detect the emitted light with a Lumitester C-100. |
| | Use a blank solution obtained by adding enzyme dilution buffer |
| | (Reagent C) instead of the sample. |

<Measurement of ATP standard solution>

| | |
|---|---|
| 1. | Add 0.1 ml of ATP standard solution (Reagent B) to 0.1 ml of |
| | CheckLite 250. |
| 2. | Detect the emitted light with a Lumitester C-100. |
| | Use a blank solution obtained by adding diluted water instead |
| | of ATP standard solution (Reagent B). |
| (2) | Thermostable luciferase |
| | The properties of thermostable luciferase used in this Example |
| | are shown in Table 3. Moreover, the standard protocol of its |
| | use and the composition of reaction reagents are shown in Table 4. |

TABLE 3
Properties of thermostable luciferase

| Properties | |
|---|---|
| Molecular weight | ca. 60 kDa (gel filtration) |
| Structure | monomer of ca. 60 kDa (SDS-PAGE) |
| Specific activity | 1.41011 RLU/mg purified protein |
| Michaelis constants | 1.9104 M (ATP) |
| | 1.5104 M (D-luciferin) |
| pH optimum | ca. 7.0-8.5 |
| pH stability | 6.0-9.0 |
| Thermal stability | at or below 40° C. |
| Stability (solution form) | stable at 25° C. for at least 5 days |

TABLE 4
Thermostable luciferase use protocol

<Reagent>

| | |
|---|---|
| A. | Tricine-NaOH buffer, 50 mM; pH 7.8: |
| | Dissolve 4.48 g of Tricine in 450 ml of distilled water, adjust |
| | to pH 7.8 with 4N NaOH, and dilute with distilled water to 500 ml. |
| B. | ATP solution, 40 mM: |
| | Dissolve 2.42 g of ATP•Na²⁺ and 896 mg of Tricine in 90 ml |
| | of distilled water, adjust to pH 7.8 with 4N NaOH, and dilute |
| | with distilled water to 100 ml. |
| C. | Luciferin solution, 5.0 mM: |
| | Dissolve 100 mg of D-luciferin in 71.4 ml of Tricine-NaOH |
| | buffer (Reagent A) and adjust to pH 7.8 with 4N NaOH. |
| D. | MgSO₄ solution, 0.1 M: |
| | 2.47 g of MgSO₄•7H₂O/100 ml of Tricine-NaOH buffer |
| | (Reagent A). |
| E. | Enzyme dilution buffer: |
| | Dissolve 4.48 g of Tricine, 185 mg of EDTA.Na²⁺•2H₂O, |
| | 31.5 μl of 2-mercaptoethanol, 25 g of glycerol, and |
| | 5 g of bovine serum albumin (BSA) in 450 ml of distilled water, |
| | adjust to pH 7.8 with 4N NaOH, and dilute with distilled water |
| | to 500 ml. |

<Sample>

Dilute the freeze-dried enzyme to a volume activity of
11031.5105 RLU/ml in ice-cold enzyme dilution buffer (Reagent E).

<Procedure>

| | |
|---|---|
| 1. | Prepare the following substrate solutions (immediately before use) |
| | 2.0 ml of Tricine-NaOH buffer (Reagent A) |
| | 0.5 ml of ATP solution (Reagent B) |
| | 2.0 ml of luciferin solution (Reagent C) |
| | 0.5 ml of MgSO₄ solution (Reagent D) |
| 2. | Add 0.1 ml of sample to cuvette. |
| 3. | Load the cuvette to a luminometer heated to 30° C. |
| 4. | Immediately after adding 0.1 ml of substrate solution, measure |
| | the amount of luminescence for 20 seconds. |
| | Use a blank solution obtained by adding enzyme dilution buffer |
| | (Reagent E) instead of the sample. |
| 3. | Method |
| | Hereinafter, specific experimental procedures will be described. |
| | The reaction cell contained template DNA, primer, DNA |
| | polymerase, PPDK, apyrase, luciferase, AMP, |
| | phosphoenolpyruvate (PEP), and luciferin. A variety of salts |
| | were additionally contained in the reaction solution. The |
| | composition of the reaction solution used is shown in a table |
| | below. |

TABLE 5
Composition of reaction solution

| Reagent | Concentration | |
|---|---|---|
| Tricine (pH 7.8) | 60 | mM |
| MgAc | 20 | mM |
| PPDK | 15.0 | U/mL |
| Luciferase | 200.0 | GLU/mL |
| Exo- Klenow | 50 | U/mL |
| Apyrase | 2 | U/mL |
| Luciferin | 0.4 | mM |
| PEP•3Na | 0.08 | mM |
| AMP | 0.4 | mM |

Four nucleic acid substrates dNTPs (dATPαS, dCTP, dGTP, and dTTP) were sequentially added one by one to the reaction cell. In this reaction, ATP plays an important role as a substrate for luminescence reaction. dATP usually used as a nucleic acid substrate for complementary strand-synthesis is structurally similar to ATP and functions as a substrate for luminescence reaction, albeit with low efficiency. Therefore, dATP generates background light and influences detection sensitivity when luminescence reaction and complementary strand synthesis reaction are performed in one reaction cell. Thus, dATPαS with exceedingly low ability as a luminescent substrate was used instead of dATP in this experiment. Other dNTPs (dCTP, dGTP, and dTTP) were used without change because they exert no influence on detection sensitivity because of their properties of poorly functioning as a luminescent substrate.

In the reaction cell, the primer is first hybridized to the single-stranded template DNA. Primer extension starts by complementary strand synthesis brought about by a nucleic-acid base (nucleic acid substrate) added to the reaction cell, if the nucleic acid substrate is complementary to the template DNA base species adjacent to the 3′ end of the primer hybridized with the template DNA. This complementary strand synthesis reaction produces PPi as a by-product. This PPi reacts with AMP and PEP in the PPDK-catalyzed reaction described above to produce ATP, pyruvate, and phosphate. ATP proceeds to react with luciferin by the action of luciferase to produce AMP, PPi, oxyluciferin, carbon dioxide, and light. The obtained luminescence can be detected with a photodiode detector or the like. The redundant nucleic acid substrates are almost completely degraded by apyrase to monophosphates in approximately 20 seconds and are not involved in subsequent complementary strand synthesis.

4. Result and Condition Optimization

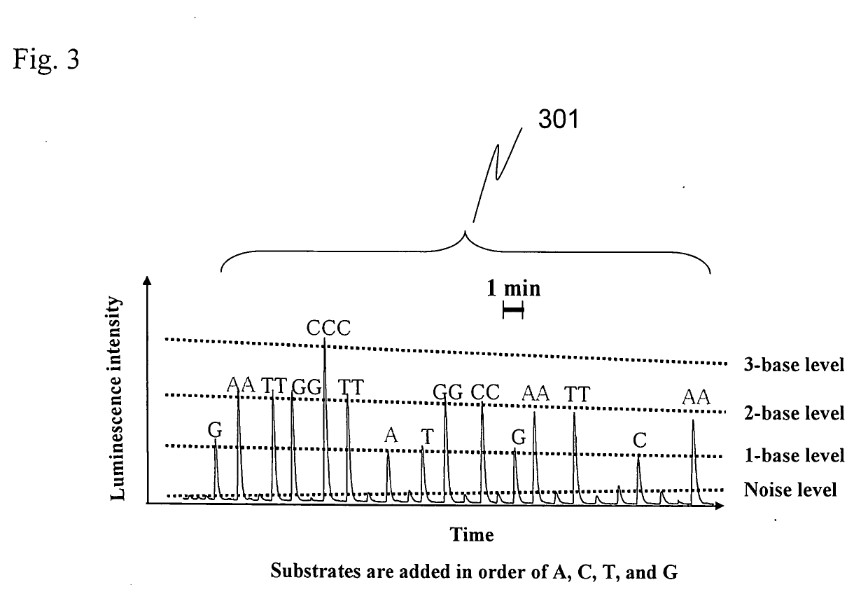

These steps are repetitively conducted in order on each nucleic acid substrate. The DNA sequence can be determined by monitoring the presence or absence of luminescence and thereby determining the base species incorporated in complementary strand synthesis. A result of sequencing using the method of the present invention is shown in FIG. 3. AMP and PPi produced in luminescence reaction are converted again to ATP by the action of PPDK. As can be seen from the diagram, a luminescence signal observed did not continue for a long time and exhibited peak forms with approximately 10-second half-width because ATP reproduced in the reaction of PPi and AMP was degraded by apyrase.

In pyrosequencing, four enzymes, DNA polymerase, apyrase, PPDK and luciferase, are allowed to simultaneously work, as described above. Since the optimum environments of these enzymes are not always identical, the conditions must be optimized for the whole system in consideration of their properties.

(1) pH

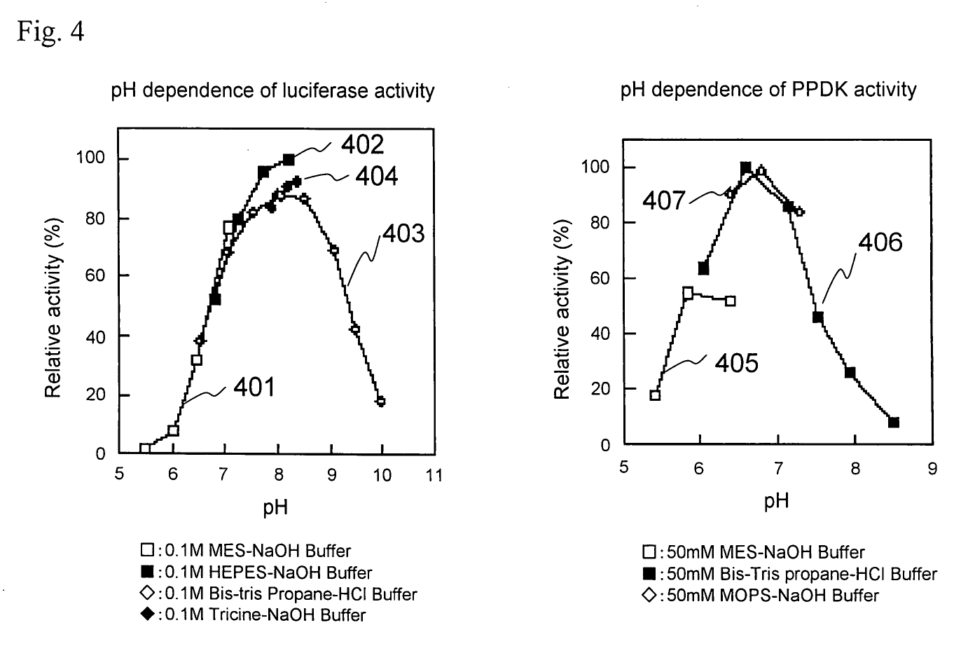

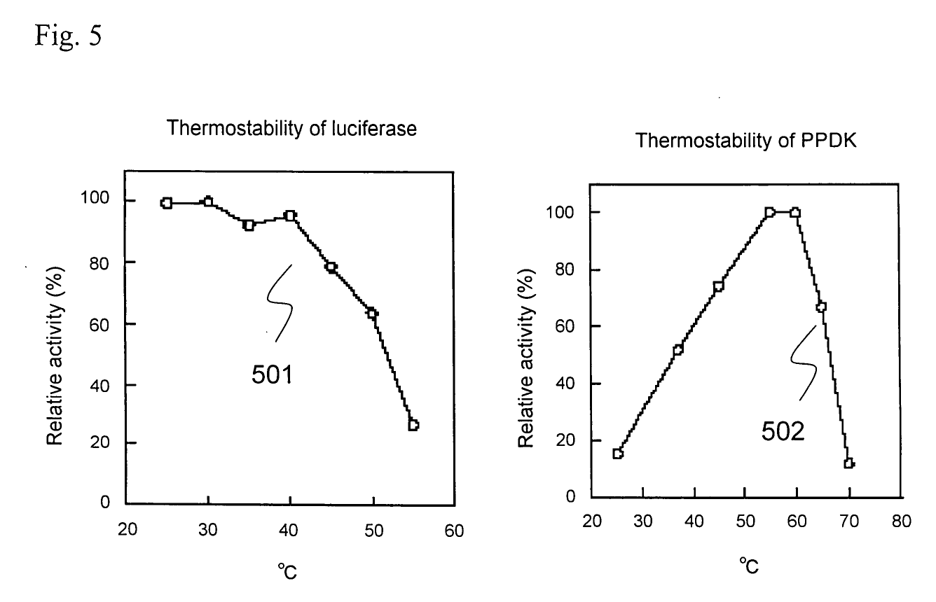

Changes in luciferase and PPDK activities depending on pH and temperature are shown in FIGS. 4 and 5, respectively. The optimum pH of luciferase was 8.0, while the optimum pH of PPDK was 6.8. As can be seen from the drawing, the PPDK activity was decreased to approximately 20% at pH 8.0. In the present invention, a key determinant of measurement precision is to balance polymerase activity described below and these PPDK and luciferase activities. Therefore, pH 7.0 to 8.0 that could be expected to provide 20% or more PPDK activity as a range free from insufficient measurement sensitivity was decided as the optimum range.

(2) Temperature

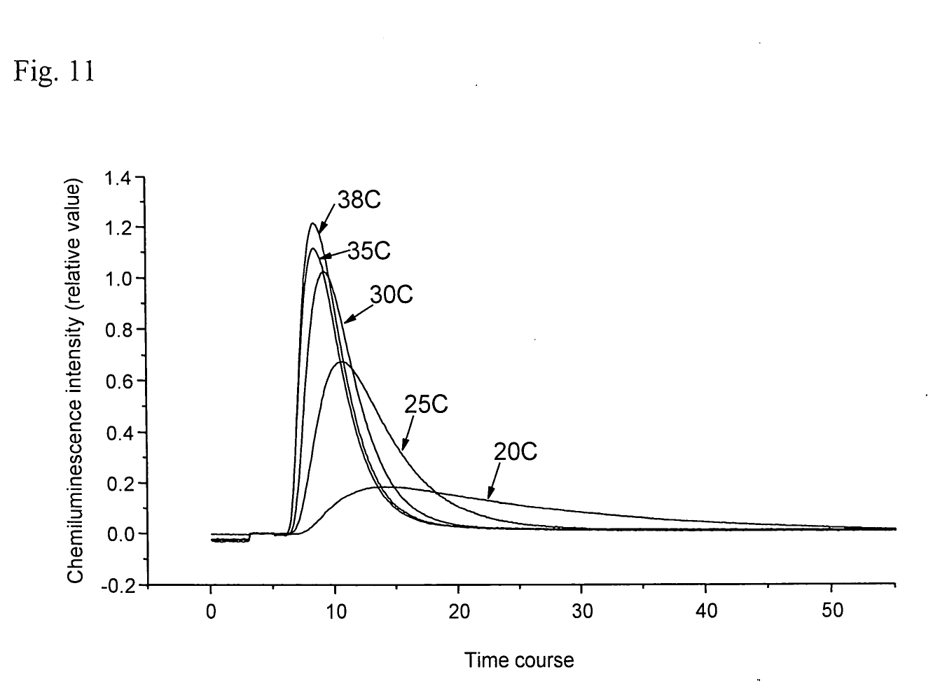

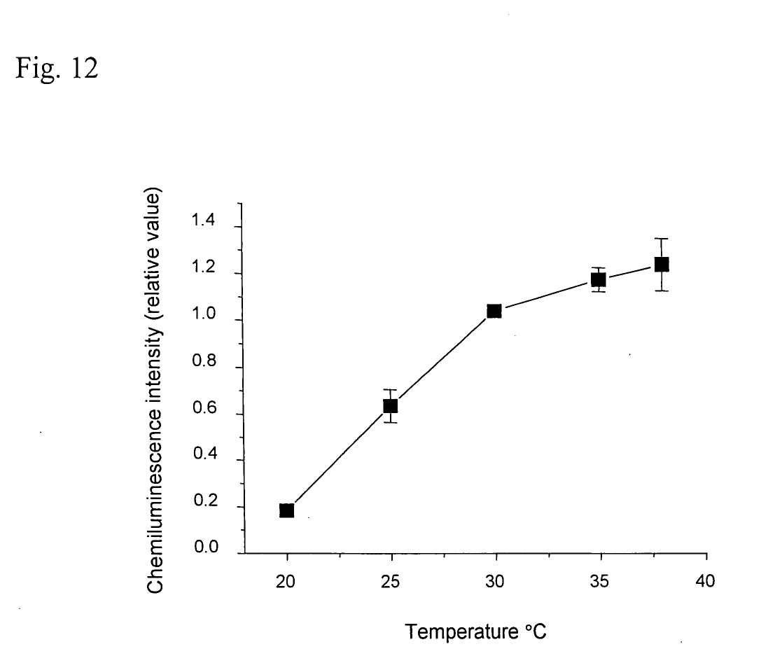

On the other hand, the PPDK activity was low at low temperatures and was rapidly decreased at high temperatures exceeding 60° C. Suitable temperatures are within the range of room temperature to 55° C., preferably 30 to 55° C., in consideration of this enzyme activity. General commercially available luciferase is deteriorated when left for a long time above 30° C. FIGS. 11 and 12 show results of measuring the amount of luminescence from nucleic acid extension preformed under each temperature condition. As can be seen from the drawings, efficient measurement can be achieved at temperatures of 30° C. or higher. The activity of the luciferase, albeit thermostable, used in this example was decreased above 40° C., as shown in FIG. 5. In consideration of this temperature dependence of PPDK and luciferase, 30 to 45° C. was decided as the optimum temperature range. The optimum temperature of typical DNA polymerase is around 37° C. and therefore falls within this range.

(3) DNA Polymerase

A variety of DNA polymerases are available in the present invention. In this Example, a Klenow fragment lacking exonuclease activity was used. If DNA polymerase used has exonuclease activity, the processes of terminal base truncation and another binding of a complementary strand base are incorporated into complementary strand synthesis procedures. Therefore, such sequencing using step-by-step complementary strand synthesis presents problems such as the repetitive reading of the same regions and the proceedings of complementary strand synthesis reaction differing from one DNA copy to another.

(4) Complementary Strand Synthesis Reaction

As described above, enzymes such as DNA polymerase, PPDK, apyrase, and luciferase are simultaneously placed in the reaction cell to perform reactions. Complementary strand synthesis is preformed by hybridizing DNA templates to primers. However, at low temperatures, hybridization occurs between the primers or between the DNA templates, or otherwise, the primer partially hybridizes to a position different from a proper hybridization position to initiate complementary strand synthesis from this wrong position. In such a case, luminescence is sometimes observed independently of the target DNA sequence and hinders sequencing. To prevent these problems, complementary strand synthesis reaction was performed at not room temperature but 37° C., at which typical luciferase activity is, however, deteriorated in a short time. Therefore, thermostable luciferase and PPDK were used in this Example. Since the priority was placed on complementary strand synthesis reaction, a pH range was set to 7.0 to 8.0, though disagreeing with the optimum pH of these enzymes.

(5) Apparatus

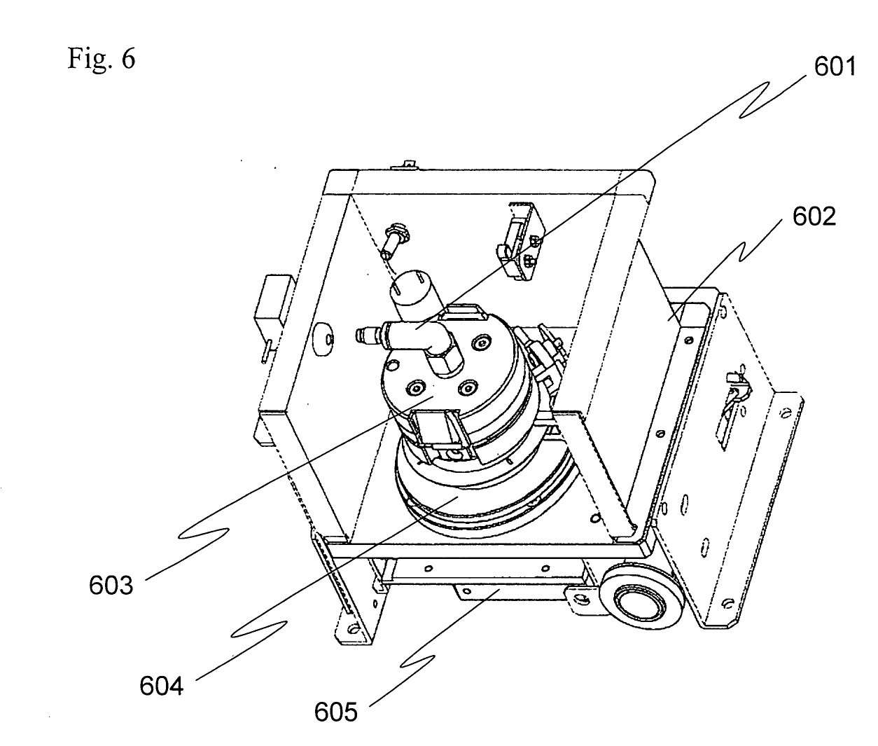

FIG. 6 shows an example of an apparatus enabling the sequencing of the present invention. As shown in FIG. 1, DNA to be sequenced, primer, complementary strand-synthesizing enzymes, AMP, PPDK, and luminescent reagents are added to a reaction cell. Nucleic acid substrates dNTPs are sequentially added from outside to the reaction cell. In this example, the nucleic acid substrates are repetitively added in the order of dATPαS→dCTP→dGTP→dTTP→dATPαS→. Each dNTP is retained in a reagent reservoir with nozzles and injected by spraying from the nozzle to the reaction cell. If the nucleic acid substrate is incorporated into the DNA strand during complementary strand synthesis, the DNA length is increased by one base while PPi is released as a by-product. This PPi is converted to ATP through a series of reactions described above, which in turn reacts with luciferin to emit light. This chemiluminescence is detected with a photodetector beneath the reaction cell. The redundant dNTPs are degraded by apyrase prior to next nucleic acid substrate injection.

(6) AMP Concentration

Since DNA complementary strand synthesis reaction is completed in a very short time, a rate-determining step of the reaction cycle is generally the process of conversion of PPi to ATP. The conventional method, which attempted to facilitate the proceeding of this process, resulted in increase in background light due to APS added in large amounts, and required approximately 1 pmol DNA sample for sequencing. On the other hand, in this Example, background light is small by virtue of the absence of APS.

However, AMP used is structurally similar to dATP used in complementary strand synthesis or to ATP serving as a luminescent substrate and, if present in large amounts, might adversely affect enzymatic reactions in the reaction cell such as complementary strand synthesis.

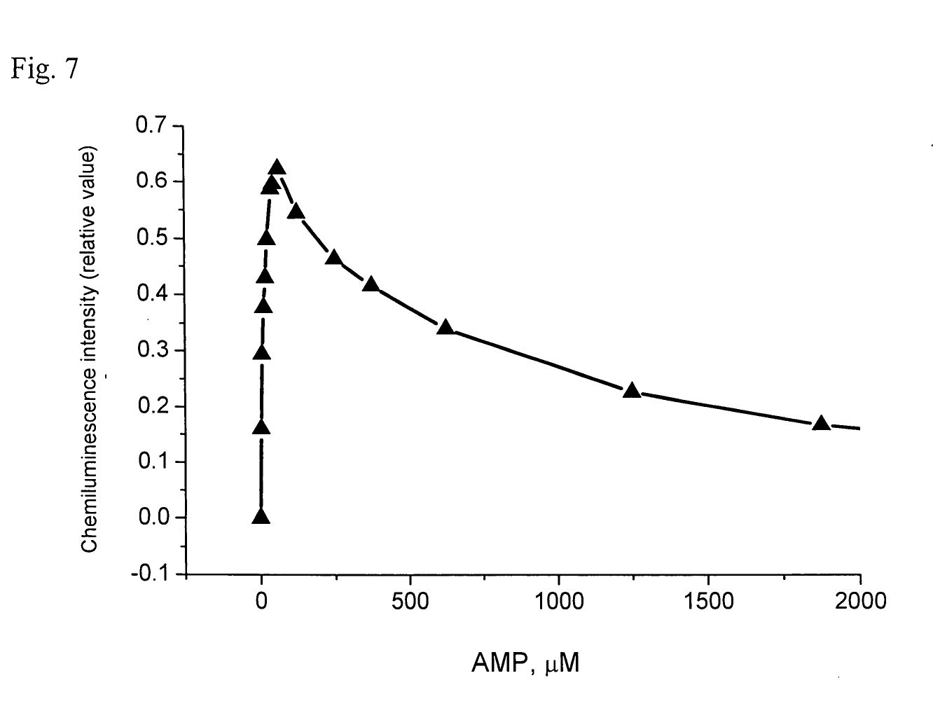

FIG. 7 shows a result of examining changes in luminescence amount obtained by adding DNA to the reaction cell in the coexistence of varying concentrations of AMP. The luminescence obtained by adding DNA (light emitted by PPi obtained in DNA complementary strand synthesis) is generated only in the presence of AMP. Since luminescence reaction was performed with ATP, luminescence was rapidly increased with increases in AMP concentration but quickly decreased after peaking in around 100 μM. AMP concentrations giving sufficient luminescence intensity are within the range of 30 to 600 μM. As is obvious, the use of AMP within this concentration range enables sequencing with 0.1 pmol DNA sample.

(7) ATP Production by AMP-PPDK System

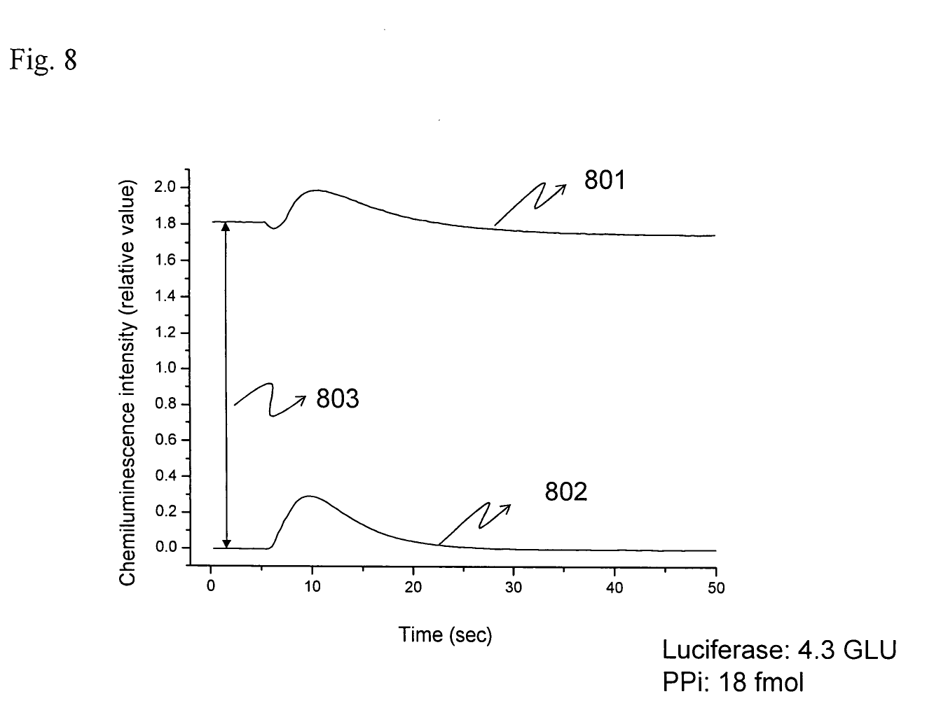

FIG. 8 shows a result of comparing obtained signal intensity and background light signal intensity between the method of the present invention using AMP and PPDK in the luciferin-luciferase reaction system and the conventional method using APS and ATP sulfurylase. As can be seen from the diagram, the system of the present invention using AMP and PPDK gave background light less by two orders of magnitude than that of the conventional system, although no significant difference in luminescence amount by PPi or ATP added in given amounts was observed between the system of the present invention and the conventional system using APS.

(8) Luciferase

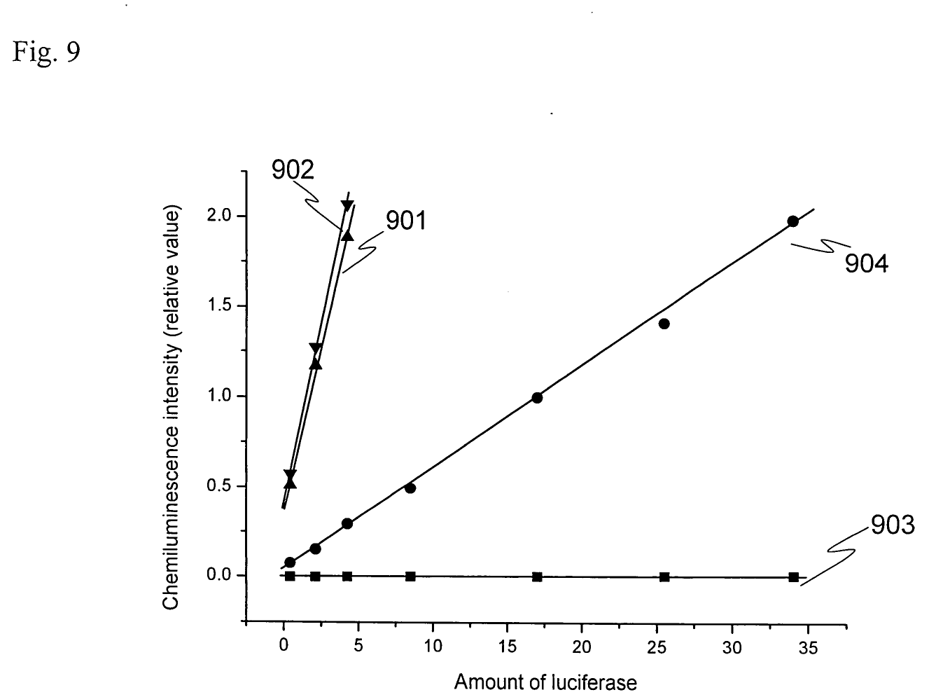

The amount of luminescence also relies on the amount of luciferase. FIG. 9 shows a result of examining background light generated from APS in the presence of varying amounts of luciferase, background light generated by the method of the present invention, and luminescence obtained by adding ATP. As the amount of luciferase is increased, the chemiluminescence signal is also increased, and highly sensitive measurement is achieved. However, in the conventional method, the amount of luciferase can not be increased because the increased luciferase amount also increases background light to a scale-over level, resulting in substantially incapable measurement. On the other hand, the method of the present invention, which generates background light in only small amounts, is capable of measurement even at high luciferase concentrations and is capable of sequencing with a trace amount of DNA.

(9) Sample Amount

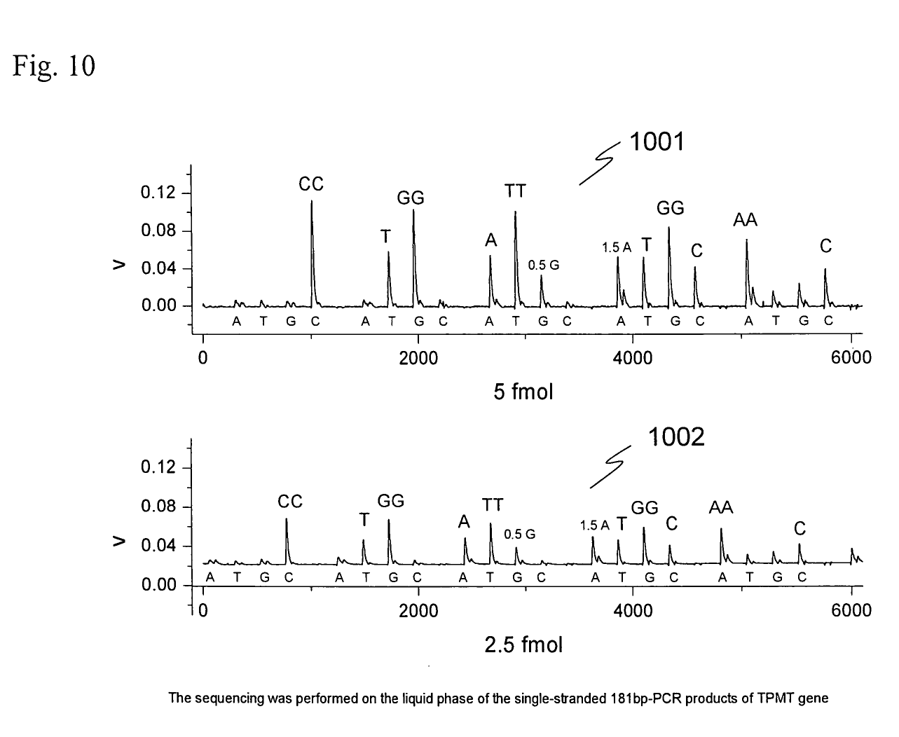

FIG. 10 shows a result of sequencing a trace amount of a DNA sample (181-base PCR amplification product of TPMT gene) by the method of the present invention. The conventional method required 0.5 to 1 pmol DNA for DNA sequencing, whereas the method of the present invention was confirmed to sequence 2.5 fmol DNA sample, which was two orders of magnitude smaller than the amount of the conventional method.

(10) PPase Addition

AMP included in reagents sometimes contains PPi as impurities, which may influence measurement. By contrast, the addition of a trance amount of PPase equal to or below 10 U/L (preferably 1 U/L) is convenient because it gradually degrades residual PPi. Although PPase also degrades PPi generated in complementary strand synthesis reaction, the degrading enzyme PPase in trace amounts does not affect DNA sequence analysis. When background light was compared between a reagent after several days without the addition of PPase and a reagent after several days with the addition of PPase, increase in background light resulting from PPi generated by dNTP degradation was observed in the reagent after several days without the addition of PPase, whereas PPi generated from the reagent after several days with the addition of PPase was decreased by the PPase addition to an ignorable degree. Namely, the addition of a trace amount of PPase was confirmed to be effective for removing PPi in reagents causing background light.

As described above, the method of the present invention enabled sequencing with a trace amount of a DNA sample by using AMP that does not serve as a luminescent substrate for luciferase in ATP production reaction and further optimizing the concentration range thereof as well as reaction pH and temperature. In the method of the present invention, the amount of a DNA sample used in sequencing is one to two orders of magnitude smaller than that of the conventional method. This suppresses the amounts of reagents used and achieves drastic reduction in sequencing cost. Furthermore, the method of the present invention can be practiced with a convenient apparatus. Therefore, cost reduction effect as the whole measurement system is much greater.

Conventional standard DNA analysis methods use gel electrophoresis with expensive apparatuses and reagents. Pyrosequencing performing step-by-step DNA complementary strand synthesis by chemiluminescence also requires expensive apparatuses and high cost required for sequencing due to large amounts of reagents used. Pyrosequencing that utilizes a convenient apparatus using inexpensive parts such as photodiode as a detection portion is also possible but remains the same in that large amounts of reagents are used. A cause of the consumption of large amounts of reagents in the conventional method is to reduce the influence of background light generated in processes uninvolved in sequencing reaction. The present invention provides a sequence analysis method without the use of such processes and achieves sequencing using the reagent amount smaller by one to two orders of magnitude than that of the conventional method, and an inexpensive apparatus. The method of the present invention provides an inexpensive, easy and simple DNA sequencing method and a kit therefor and brings immeasurable benefits to bio-related fields. The application scope thereof is not limited to DNA sequencing and is as divers as genetic testing, gene expression analysis with mRNA, food inspection, and bacteriological examination.

What is claimed is:

1. A method for nucleic acid analysis comprising the steps of
subjecting a reaction solution comprising a sample nucleic acid to complementary strand synthesis with the sample nucleic acid as a template,
reacting pyrophosphate produced in the complementary strand synthesis with 30 to 800 μM AMP in the coexistence of pyruvate phosphate dikinase to produce ATP,
performing luciferase reaction with the ATP as a reaction substrate, and
detecting chemiluminescence generated in the luciferase reaction to determine the presence or absence of the complementary strand synthesis.

2. The method for nucleic acid analysis according to claim 1, wherein the method performs, at the complementary strand synthesis step, complementary strand synthesis by sequentially adding one by one four nucleic acid substrates or derivatives thereof corresponding to bases A, G, T, and C, and sequences the sample nucleic acid on the basis of the presence or absence of the complementary strand synthesis.

3. The method for nucleic acid analysis according to claim 1, wherein the method performs, at the complementary strand synthesis step, complementary strand synthesis by simultaneously adding two or more of four nucleic acid substrates or derivatives thereof corresponding to bases A, G, T, and C, and sequences the target site on the basis of the presence or absence of the complementary strand synthesis.

4. The method for nucleic acid analysis according to claim 1, wherein the redundant nucleic acid substrates or derivatives thereof are enzymatically degraded.

5. The method for nucleic acid analysis according to claim 1, wherein the pyruvate phosphate dikinase and/or the luciferase are thermostable enzymes that stably function at 40° C. or higher.

6. The method for nucleic acid analysis according to claim 1, wherein the reaction solution has a pH of 7.0 to 8.0.

7. The method for nucleic acid analysis according to claim 1, wherein the reaction solution has a temperature of 30 to 45° C.

8. The method for nucleic acid analysis according to claim 1, wherein the reagents are treated in advance with enzyme(s) that digest pyrophosphate and/or ATP.

9. The method for nucleic acid analysis according to claim 8, wherein the enzyme is pyrophosphatase.

10. A kit for nucleic acid analysis comprising
1) DNA polymerase,
2) four nucleic acid substrates or derivatives thereof corresponding to bases A, G, T, and C,
3) AMP, phosphoenolpyruvate, and pyruvate phosphate dikinase, and
4) luciferase and luciferin,
wherein the AMP has a concentration of 30 to 800 μM in a reaction solution and is intended for reaction with pyruvate phosphate dikinase in the reaction solution.

11. The kit for nucleic acid analysis according to claim 10, wherein the pyruvate phosphate dikinase and/or the luciferase are thermostable enzymes that stably function at 40° C. or higher.

12. The kit for nucleic acid analysis according to claim 10, wherein the kit further comprises an indication stating that the reaction solution is adjusted to pH 7.0 to 8.0.

13. A kit for nucleic acid analysis comprising
1) DNA polymerase,
2) four nucleic acid substrates or derivatives thereof corresponding to bases A, G, T, and C,
3) AMP, phosphoenolpyruvate, and pyruvate phosphate dikinase, and
4) luciferase and luciferin,
wherein the kit comprises an indication stating that the AMP is reacted at a concentration of 30 to 800 μM in a reaction solution with pyruvate phosphate dikinase.

14. The kit for nucleic acid analysis according to claim 13, wherein the pyruvate phosphate dikinase and/or the luciferase are thermostable enzymes that stably function at 40° C. or higher.

15. The kit for nucleic acid analysis according to claim 13, wherein the kit further comprises an indication stating that the reaction solution is adjusted to pH 7.0 to 8.0.
